(19) United States
(12) Patent Application Publication
(10) Pub. No.: US 2019/0071783 A1
(43) Pub. Date: Mar. 7, 2019

(54) ELECTROCHEMICAL CELL FOR GAS-PHASE REACTANT IN LIQUID ENVIRONMENT

(71) Applicant: HPNOW APS, Copenhagen (DK)

(72) Inventors: Arnau VERDAGUER CASADEVALL, Kobenhavn (DK); Rasmus FRYDENDAL, Bronshoj (DK)

(21) Appl. No.: 16/084,587

(22) PCT Filed: Mar. 10, 2017
(86) PCT No.: PCT/EP2017/055710

§ 371 Date: Sep. 13, 2018

Related U.S. Application Data
(60) Provisional application No. 62/309,655, filed on Mar. 17, 2016.

(51) Int. Cl. C25B 9/10; C25B 1/30; C25B 15/08; C25B 9/18; C25B 11/03; C02F 1/467; C02F 1/461; A61L 2/20; A61L 2/26; A61L 9/04; A61L 9/12

(52) U.S. Cl. CPC C25B 9/10; A61L 2202/15; C25B 15/08; C25B 9/18; C25B 11/035; C02F 1/4672; C02F 1/46104; A61L 2/208; A61L 2/26; A61L 9/046; A61L 9/12; C02F 2201/46115; A61L 2209/135; A61L 2202/11; C25B 1/30

(57) ABSTRACT

The invention is directed to an electrochemical cell for production of chemicals from a gas-phase reactant in a liquid environment. The electrochemical cell has a membrane electrode assembly configuration and is comprised of an anode, an ion exchange membrane and a cathode. The membrane electrode assembly is in direct contact with a liquid solution to facilitate extraction and handling of the produced chemicals, and the gas reactant is delivered through the liquid to the membrane electrode assembly. The present invention relates to a process and electrochemical cell for use in the synthesis of chemicals. The present invention relates to the electrocatalysts used and their incorporation into a membrane electrode assembly, MEA. The present invention also relates to the electrodes used in a membrane electrode assembly, in particular the gas diffusion layer, and a method for transport of gas-phase reactants into a liquid-immersed membrane electrode assembly.

The invention is related to an electrochemical cell with improved properties for production of chemicals from a gas-phase reactant in a liquid environment. The electrochemical cell has a membrane electrode assembly configuration and is comprised of an anode, an ion exchange membrane and a cathode. The membrane electrode assembly is in direct contact with a liquid solution to facilitate extraction and handling of the produced chemicals, and the gas reactant is delivered through the liquid to the membrane electrode assembly.

The present invention also relates to a novel process and electrochemical cell for use in the synthesis of chemicals. The present invention relates to the electrocatalysts used and their incorporation into a membrane electrode assembly, MEA. The present invention also relates to the electrodes used in a membrane electrode assembly, in particular the gas diffusion layer, and a method for transport of gas-phase reactants into a liquid-immersed membrane electrode assembly.

### BACKGROUND OF THE INVENTION

Electrochemical methods are a viable strategy to synthesize chemicals on-site, on-demand using compact reactors. For example, they are employed today for the synthesis of chlorine-based compounds (e.g. sodium hypochlorite) used in water treatment and biocide applications, or for the synthesis of CO from CO₂.

Typically electrochemical cells are composed of three parts: anode, electrolyte and cathode. An oxidation reaction takes place at the anode, while charged species are transported through the electrolyte and a reduction reaction happens at the cathode.

Of particular interest is the development within the last decades of electrolytes based on ion exchange membranes. These substitute liquid electrolytes, which require extensive handling, can be hazardous and ultimately complicate the separation of generated products. The term electrochemical cell in this document refers to a membrane electrode assembly, whereby the electrolyte is an ion exchange membrane. Standard techniques to build membrane electrode assemblies including catalyst-coated membranes and gas diffusion electrodes are well-known to practitioners in the field (see for example Handbook of Fuel Cells: Fundamentals, Technology and Applications, Wiley VCH, 2014). Membrane electrode assemblies can be comprised of more than one membrane and more than one gas diffusion layer on either side.

The use of ion exchange membranes as electrolytes is widespread for energy applications, for instance in fuel cells and electrolyzers, but thus far has seen limited use for the synthesis of chemicals despite allowing easy separation of reaction products. One of the reasons for such limited use is that chemicals synthesized at the catalyst layer of the electrochemical cell need to be extracted from the membrane electrode assembly. This is particularly challenging when gas-phase reactants need to be fed into the cell, and most solutions proposed in the literature rely purely on diffusion of reactants and products (see for example Handbook of Fuel Cells: Fundamentals, Technology and Applications, Wiley VCH, 2014).

To facilitate the extraction of products some practitioners have turned into immersing the electrodes into liquid solutions. That way, products dissolve into the liquid and diffuse through it. This is widely practiced in liquid electrolytes (see for example U.S. Pat. No. 6,712,949, Gopal), but not utilized in membrane-based electrochemical cells, despite the advantages these electrodes represent in terms of scalability and product separation.

In membrane electrode assembly configurations products formed in the vicinity of the ion exchange membrane need to be extracted through the gas diffusion layer. Particles, for instance from catalyst dispersions, can be present in the gas diffusion layer and decompose products on their way out by chemical or electrochemical means.

One of the key reasons why membrane electrode assemblies are not utilized in a liquid environment is that gas reactants, such as oxygen, hydrogen, CO or CH4 have very low solubility in most liquids, including aqueous solutions. For example, oxygen solubility in water is only of 40 mg/L under normal conditions. In turn, this restricts availability of reactants at the electrode and achievable current densities, which are typically limited to a few mA per cm². This is exemplified in the report by Li et al (Drinking Water Purification by Electrosynthesis of Hydrogen Peroxide in a Power-Producing PEM Fuel Cell, ChemSusChem 2013), where current densities reach values up to 30 mA/cm².

Yamanaka et al. report an attempt to mitigate the problem addressed at oxygen reduction to hydrogen peroxide (Neutral H2O2 Synthesis by Electrolysis of Water and O2, Angewandte Chemie 2008). In this work, half of the electrochemical cell cathode is exposed to gaseous oxygen, and the other half is immersed in liquid electrolyte. Using this method current densities of 20 to 60 mA/cm² can be achieved. However, faradaic efficiency to hydrogen peroxide is 26%, which could indicate that hydrogen peroxide removal from the electrode is difficult.

The usual method adopted by practitioners in the art to solve the problems associated with the limited mass transport of gasses in liquids is to operate the electrochemical cells in a fully gaseous environment, with limited liquid interactions.

Examples of this strategy for the production of chemicals are found in GB application 2012/052316. In this document a fuel cell aimed at producing hydrogen peroxide is described. Hydrogen peroxide is produced at the cathode of the reactor using pressurized humidified oxygen, without the presence of liquid water (other than water from the membrane or produced at the catalyst). Current density in this case approaches 80 mA/cm², but faradaic efficiency to hydrogen peroxide is not stated.

A similar example is U.S. Pat. No. 7,892,408 B2, disclosing an electrolyzer cell for hydrogen peroxide production using gaseous oxygen or air feeds to the cathode, without the presence of liquid water. Current densities in this case are 200-300 mA/cm².

### SUMMARY OF THE INVENTION

The present invention was made in view of the prior art described above, and its objective is to facilitate electrochemical production of chemicals in a membrane electrode assembly configuration. Using this invention it is possible to enhance extraction of products from the membrane electrode assembly. The invention also enables an improved utilization of gaseous reactants.

In a first aspect, the present invention provides enhanced mass transport of gas phase reactants to the electrochemical cell and easy extraction of products. This is very useable for the electrochemical synthesis of products from a gaseous reactant in a membrane electrode assembly type electrochemical cell. The gas is delivered to the electrochemical cell by convection through the liquid solution and the gas diffusion layer.

The presence of a liquid solution in immediate contact with the anode and/or cathode of the cell eases extraction of products by facilitating their diffusion to the liquid.

In a second aspect, the invention provides a catalyst-coated membrane with improved properties for hydrogen peroxide generation. This allows for a very thin catalyst layer adjacent to the membrane, which decreases residence time of products in the catalyst layer and facilitates their extraction.

In a third aspect, the invention provides a way to optimize gas usage in one or several electrochemical cells.

In a fourth aspect, the electrochemical cells generate hydrogen peroxide by cathodic oxygen reduction, and the generated hydrogen peroxide is introduced into a water line for uses including but not limited to disinfection and bleaching. Hydrogen peroxide generated using this method can also be vaporized for room or surface disinfection, or combined with other disinfection methods such as ultra-violet light or ozone for enhanced disinfection properties.

### DETAILED DESCRIPTION OF THE INVENTION

The present invention relates to a novel electrochemical cell design in the membrane electrode assembly configuration and a method for improved transport of reactants. A membrane electrode assembly consists of a cathode and an anode which are pressed against either sides of a membrane, shown in FIG. 1. Point number 1 is the membrane electrode assembly. Point number 2 shows the anode gas diffusion layer or current collector, point number 3 the anode catalyst layer. Together these form the anode of the cell. Point number 4 is the ion exchange membrane, preferably a cation exchange perfluorinated membrane. Point number 5 is the cathode catalyst layer, and point number 6 the cathode gas diffusion layer or current collector. Together these form the cathode of the cell. In particular the present invention is directed to a method for improved mass transport of reactants and products in an electrochemical cell. The invention is also related to a method allowing easy extraction of products generated at the electrodes. The invention is further related to optimized use of a gaseous reactant.

Accordingly, the present invention provides a liquid solution in the immediate vicinity of the electrodes to facilitate extraction of the products from the electrochemical cell. The liquid solution can be static or flowing, preferably flowing. The liquid solution is preferably composed of more than 90% of water. The temperature of the liquid solution is between −20 and 500° C., preferably between 0 and 100° C.

The invention also provides a method to deliver a gaseous reactant through the liquid solution and into the electrode in a manner that gas solubility and diffusion in the liquid is not limiting current density in the cell. The gas is delivered to the electrochemical cell through a gas disperser. An example of a suitable gas disperser could be a tube or other suitable gas conduit, which may have a suitable fluidizing media in contact with the liquid solution or electrochemical cell to facilitate dispersion of the gas. Preferably, the gas disperser is placed between 0 and 1 cm of the electrode, even more preferably between 0 and 0.3 cm, in a manner that gas is directed into the electrode. Using this invention, gas diffusion in the liquid is overcome by convection and mass transport of reactants is enhanced. Suitable gas reactants are well known to those versed in the art, several examples include hydrogen, carbon monoxide, carbon dioxide, methane, propene, oxygen and others. The preferred electrode area covered by the gas disperser is between 10 and 90%, even more preferred between 30 and 70%.

The present invention also relates to a device for the electrochemical production of chemical compounds in an electrochemical cell consisting of a membrane electrode assembly. The membrane electrode assembly is at least partly immersed in a liquid solution, at least on the side of the membrane requiring a gas reactant. For clarity, the membrane electrode assembly may be partly or fully immersed in the liquid solution. FIG. 2 shows a diagram of the gas delivery system onto half a membrane electrode assembly, point number 7. Point number 8 shows the gas disperser, preferably delivered through a porous material which allows gas to cross into the liquid layer, depicted in number 9. The gas bubbles formed go on to the gas diffusion layer, 5, and into the catalyst layer, 6. 4 represents the ion exchange membrane separating anode and cathode. An advantage of the present invention over prior art is the enhanced mass transport of gaseous reactants through a liquid solution, which enables substantially higher current densities at the electrode, as shown in FIG. 3, while the products can be easily carried away by the liquid. The electrodes of the membrane electrode assembly can be catalyst-coated membranes or gas diffusion electrodes or a mixture of the two. As an example, one could have a membrane electrode assembly with a gas diffusion electrode on the cathode side and a catalyst-coated membrane on the anode side.

FIG. 4 shows a frame containing gas dispersers that can be coupled to an electrochemical cell. The frame, marked by point 10, is to be pressed against one side of a membrane electrode assembly, and it has a volume available for a liquid and gas delivery channels. The structures marked by point 11 host the gas dispersers and may contain a piece of porous material or other suitable fluidizing media to enhance dispersion of the gas. With a slight gas overpressure, the gas is driven through the porous material and directed into the gas diffusion layer in a highly efficient manner. The frame indicated by point 12 is used on the other side of the electrochemical cell. This frame contains structures, point 13, for counter pressure and may or may not include the gas delivery system described above. FIG. 5 shows a side view diagram of a gas delivery system. Point 14 indicates a volume for liquid, point 1 the membrane electrode assembly and point 15 a gas disperser. The gas disperser could be a tube or it could also contain one or more porous layers to act as fluidizing media. These are helpful to facilitate dispersion of the gas into the liquid. In this configuration the MEA can be partly or fully immersed in liquid for optimal extraction of generated products while the gas channel in close vicinity to the MEA provides improved gas delivery.

Another aspect of the invention, which could be combined with the method allowing for improved gas delivery, is the utilization of a gas diffusion layer facilitating extraction of the products generated at the electrode. At least one of the gas diffusion layers in the membrane electrode assembly is made of carbon, titanium or other suitable materials, and presents openings (for example through holes) in a pattern, which can be regular, semi-regular or random. The shape of the holes is not of importance to the present invention, and their dimensions are preferably between 1 μm and 5000 μm. FIG. 6 shows a schematic representation of the holes in the gas diffusion layer, depicted as a manner of example in a circular shape. A gas diffusion layer with this hole distribution facilitates extraction of the products generated at the electrode by providing an obstacle-free path to the molecules. This gas diffusion layer can be combined with a gas diffusion electrode, a catalyst-coated membrane or both. The gas diffusion layer, either as received or modified in certain ways, can be further treated to modify its interaction with water. For example, hydrophobicity can be promoted by adding polytetrafluoroethylene (PTFE) or other fluorocarbon polymers (e.g. polyvinylidene fluoride or polyvinylfluoride) in a way well-known to practitioners in the art. Hydrophilicity can be promoted in a similar manner by adding hydrophilic molecules.

Another strategy to increase removal efficiency of products from the catalyst layer, which could be combined with the previously described inventions, is to selectively deposit catalyst on a gas diffusion layer (forming a gas diffusion electrode) or on a membrane (forming a catalyst-coated membrane) in selected regions to facilitate extraction of the products generated at the electrode. This can also decrease residence time of products at the gas diffusion layer, minimizing their possible decomposition by the electrode. This selective coating can be achieved for instance by using a pattern when depositing the catalyst ink. As an example, the pattern could have between 5 and 80% of open area and a distance between coated areas between 10 and 10000 μm.

In yet another aspect of the invention, the catalyst is applied as a catalyst coated membrane. That way, catalyst material is concentrated in the vicinity of the membrane and has good ionic contact. Importantly, using this method the catalyst layer is thin, which is favorable because it minimizes decomposition of chemical compounds produced at the catalyst during the extraction process. Using a catalyst coated membrane for the production of chemical compounds, especially if such compounds are in the liquid form, is beneficial to decrease residence time of the compounds in the catalyst layer, and it also allows for improved catalyst utilization and reduced loadings. A schematic representation of a catalyst coated membrane and a gas diffusion electrode is shown in FIG. 7. The catalyst coated membrane contains a suitable catalyst for the reaction. For example, if the reaction is oxygen reduction to hydrogen peroxide said catalyst could be silver, carbon, gold, Pt—Au, Pd—Au, Pt—Hg, Pd—Hg, Ag—Hg, Cu—Hg, Co-containing materials, other carbon-based structures, graphene or graphene-based compounds doped with transition metals (including but not limited to iron, nickel, cobalt, manganese, copper, silver, gold, platinum, palladium, iridium, ruthenium) or post-transition metals, porphyrins containing transition metals (including but not limited to iron, nickel, cobalt, manganese, copper, silver, gold, platinum, palladium, iridium, ruthenium) or post-transition metals or any combinations thereof. A catalyst coated membrane could also be combined with a gas diffusion electrode. The gas diffusion electrode can be catalyst-coated on either or both sides. In that manner, the catalyst layer is facing the membrane, the liquid solution or both.

FIG. 8 shows a method to prepare a catalyst coated membrane. This method consists of three steps: first, preparing a mixture of catalyst ionomer to form a catalyst ink; second, applying the catalyst ink to the ion exchange membrane to form a catalyst layer; third adding gas diffusion layers to both sides of the membrane and applying a heat and pressure treatment to facilitate bonding of the gas diffusion layers to the membrane. Preferably, the temperature applied during the third stage is between 60 and 200° C., and the pressure between 20 and 2000 kg/cm².

### BRIEF DESCRIPTION OF THE DRAWINGS

FIG. 1 represents a schematic diagram of an electrochemical cell as a membrane electrode assembly.

FIG. 2 shows a schematic diagram of the electrochemical cell design with liquid between the gas disperser and the gas diffusion layer. The gas disperser provides reactants to the catalyst layer by convection of the gas.

FIG. 3 shows experimental results for a membrane electrode assembly producing hydrogen peroxide by oxygen reduction at the cathode. The current densities for the same membrane electrode assembly with dissolved oxygen, and using the present invention are shown.

FIG. 4 is a schematic representation of a gas delivery system to the membrane electrode assembly. The frame indicated as point 10 is mounted against a membrane electrode assembly, which is then pressed from the other side with a similar frame, point 12, that may or may not include gas delivery system. The gas is delivered directly into the electrodes through gas dispersers indicated by point 11, and a pillar on the opposite side provides for counter-pressure (point 13).

FIG. 5 is a schematic showing the concept of a gas delivery system enclosing a membrane electrode assembly. Point number 14 indicates a volume used for either a flowing or stagnant liquid, point 15 indicates the gas disperser through a channel which may have fluidizing media at the end and point 1 indicates the membrane electrode assembly immersed in the liquid.

FIG. 6 shows a schematic representation of a gas diffusion layer with openings featured in the present invention.

FIG. 7 shows a schematic diagram for two membrane electrode assemblies prepared on two different ways. A gas diffusion electrode (prior art) and a catalyst coated membrane, disclosed in the current invention.

FIG. 8 is a block flow diagram explaining how to prepare a catalyst coated membrane.

FIG. 9 is a diagram that shows how the gas can be connected to one or more electrochemical cells in series.

FIG. 10 is a diagram that shows how the gas can be recirculated in one or more electrochemical cells. With this method the gas can be delivered in any stoichiometry while controlling the utilization.

FIG. 11 is a diagram showing how locally generated hydrogen peroxide can be added to a water line or reservoir for local use. Point number 16 indicates the hydrogen peroxide generator, consisting of the electrochemical cell described herein. Point 17 indicates the water input to the electrochemical cell, point the water line containing hydrogen peroxide, point 19 a reservoir for storage of the hydrogen peroxide solution and point 20 an ultra-violet light or ozone source which may or may not be present.

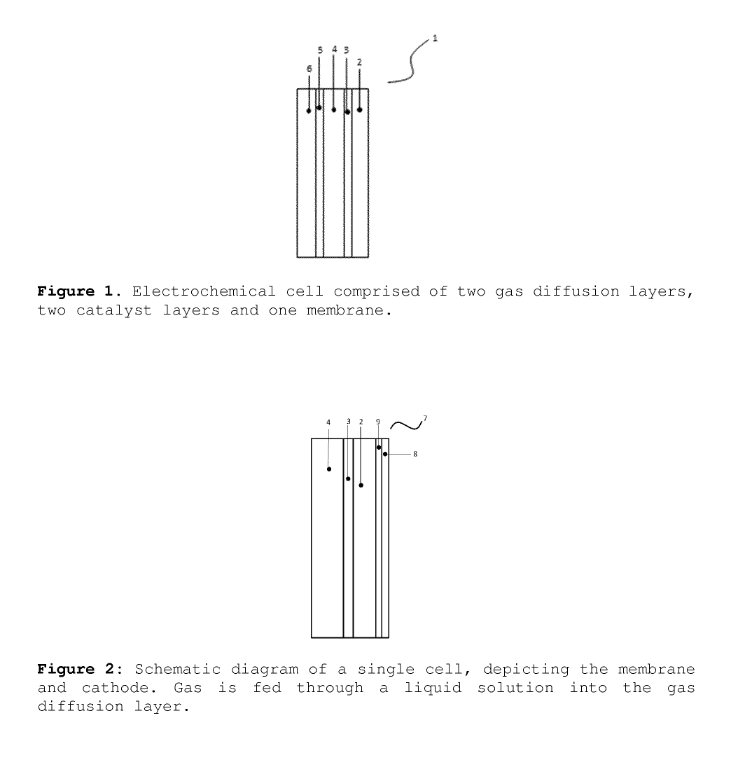

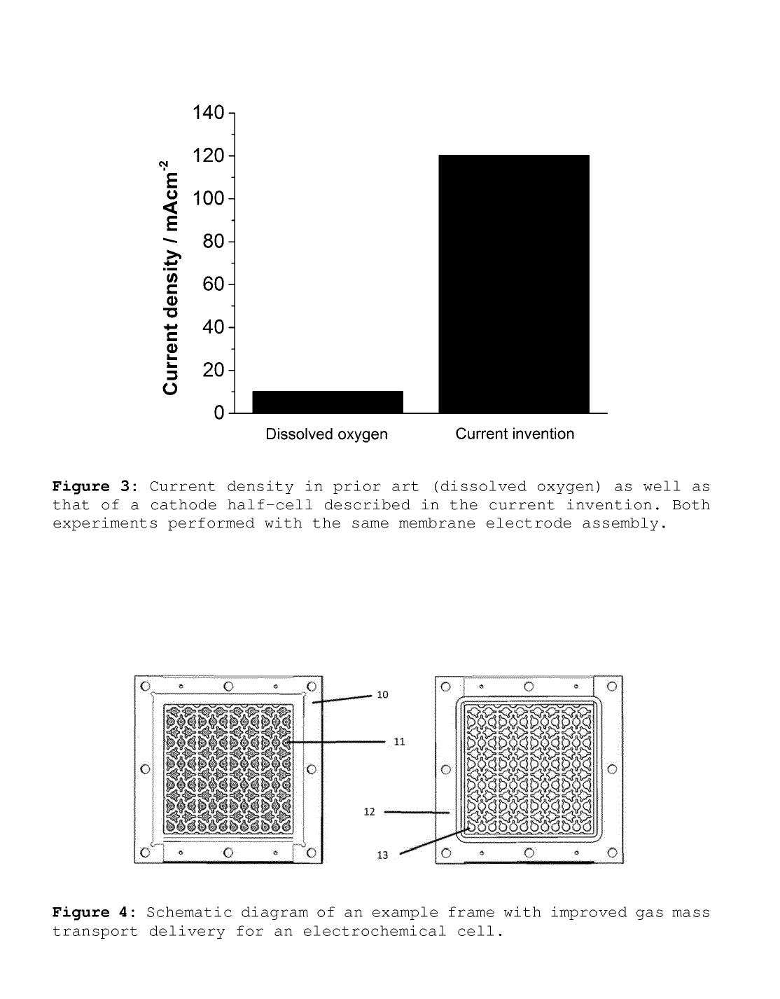

### EXAMPLES

### Example 1

Electrochemical synthesis of hydrogen peroxide

Electrochemical synthesis of hydrogen peroxide is promising for distributed generation of the chemical closer to the point of use. This would overcome limitations of the current method for industrial hydrogen peroxide synthesis, the anthraquinone process, a large-scale process, which can only be efficiently conducted at large scale chemical plants. From the production plant hydrogen peroxide is concentrated to 30-70% solutions (labelled as a hazardous material) and transported to the point of use, where it typically needs to be diluted to manageable concentrations. These could be in the ppm to few-% range. The transportation and dilution process results in extensive logistics and complexity for end-users, who despite the attractive properties of hydrogen peroxide as an effective and environmentally friendly chemical turn to other alternatives such as chlorine or formaldehyde for their processes. A method to generate hydrogen peroxide on-site would simplify use of the chemical by eliminating the logistics burden, and facilitate application of hydrogen peroxide directly at the required low concentrations and at high purity. This would also improve safety of the processes as the concentration of hydrogen peroxide would not exceed dangerous levels. Hydrogen peroxide synthesized this way could also be used to enhance dissolved oxygen levels in water or other liquids, while accurately controlling concentration.

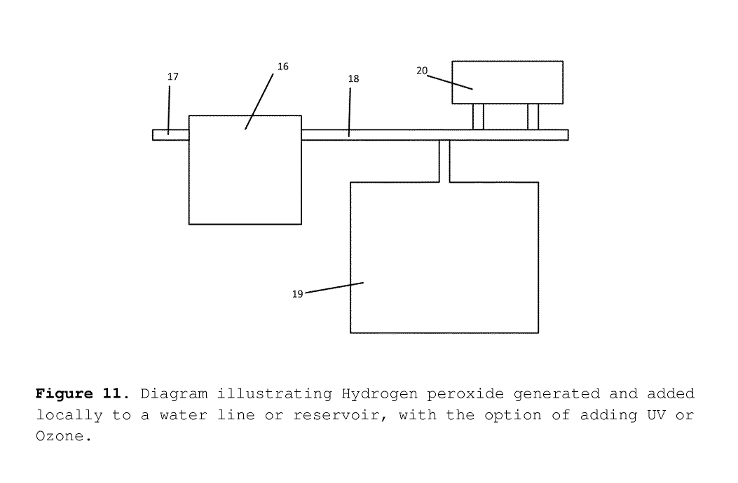

Some applications where hydrogen peroxide is used, and where its distributed generation can provide strong benefits, include bleaching in the pulp and paper industry, as a biocide or disinfectant in the agricultural and food industries (in aquaculture, animal drinking water, irrigation water treatment), in municipal and residential water treatment, in waste water treatment, in soil remediation, in swimming pools, or in healthcare for personal hygiene and as a surface and room disinfectant. Hydrogen peroxide produced locally can be introduced directly into a water line or stored in a reservoir prior to introduction into a water line, as shown in FIG. 11. Further, in some of these applications, such as water treatment, hydrogen peroxide synthesized on site can also be combined with other reagents, such as ozone, or with ultra-violet radiation, which facilitate the formation of hydroxyl radicals with even stronger oxidizing potential. This prospect is illustrated in

FIG. 11. This would be very useful for disinfection applications, since radicals are known to be more powerful disinfection agents than hydrogen peroxide itself. Such an on-site hydrogen peroxide generator, possibly combined with other reagents such as ozone or ultra-violet light, can be used in swimming pools, room disinfection, laundry facilities and water treatment facilities. In some of these applications a sensoring option can be included to facilitate hydrogen peroxide generation in accordance with necessary concentrations levels further downstream. With suitable sensoring options, for instance by Oxidation Reduction Potential or hydrogen peroxide sensors, a feedback mechanism could be implemented to control the throughput of the hydrogen peroxide generators.

Hydrogen peroxide can be synthesized on-site through an electrochemical process using water and oxygen as reactants. Electrochemical generation can be achieved in compact devices generating on-site, on-demand hydrogen peroxide directly at the diluted concentrations required. Such a device has three distinct parts: anode, membrane and cathode. Water oxidation takes place at the anode, resulting in oxygen and protons. The protons are transported through an ion exchange membrane to the cathode, where they recombine with oxygen to yield hydrogen peroxide at the surface of an appropriate catalyst. The ion exchange membrane is typically a polymer electrolyte membrane, but could also be a hydrocarbon membrane or other suitable cation exchange membranes.

The half-cell reactions are as follows:

Anode: 2H₂O→O₂+4H⁺+4e⁻

Cathode: 2O₂+4H⁺+4e⁻→2H₂O₂

It is important to minimize hydrogen peroxide decomposition, which can take place chemically or electrochemically:

2H₂O₂→2H₂O+O₂

H₂O₂+2H⁺+2e⁻→2H₂O

Other proton sources than water can be used at the anode, for example hydrogen, methane, methanol etc. These are evident to those skilled in the art.

Using the present invention in an electrochemical cell for hydrogen peroxide synthesis shows improved properties. The presence of a liquid near and through the cathode gas diffusion layer decreases residence time of hydrogen peroxide in the vicinity of the electrode, which minimizes its decomposition. Simultaneously, an oxygen containing gas is delivered to the cathode electrode, as depicted in FIG. 2 and FIG. 4. This induces fast movement of both liquid and gas species in the vicinity of the catalyst layer, which favors fast mass transport conditions. These are key to two goals: a) effective transport of oxygen to the electrode; b) decrease the residence time of the produced hydrogen peroxide at the electrode, minimizing its decomposition either by chemical or electrochemical means.

In addition, if liquid is flowing near and/or through the gas diffusion layer it further favors fast mass transport, and it is preferred over non-flowing liquid. Preferably the liquid is composed of more than 90% of water, and even more preferably it is water.

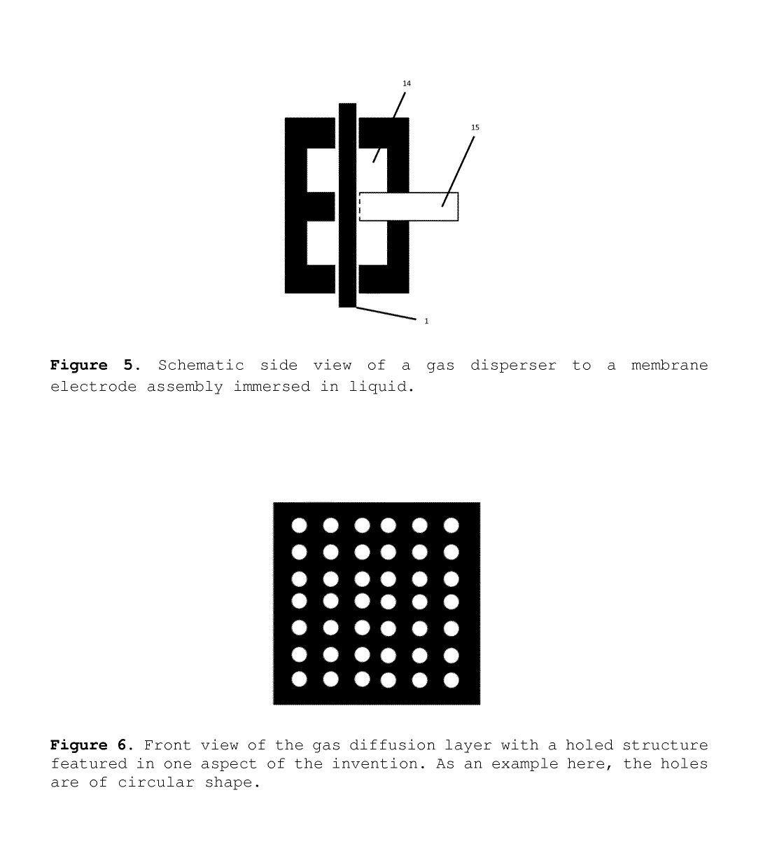

In another aspect of the invention, air (or another suitable oxygen source) is fed to the cathode side of the cells in the close vicinity, in a manner that gas is directed into the gas diffusion layer and to the catalyst layer, depicted in FIG. 2 and FIG. 5. This enhances mass transport of oxygen species to the cathode side of the cell and facilitates high current densities.

In yet another aspect, the gas diffusion layer used at the cathode side is modified to facilitate extraction of the produced hydrogen peroxide, shown in FIG. 6. These holes facilitate the transport of hydrogen peroxide out of the catalyst and gas diffusion layers because the dense carbon fiber network forming the gas diffusion layer has been removed. In addition, the holes are easily filled up with liquid which is also beneficial for removal of hydrogen peroxide. A gas diffusion layer patterned in this manner could be coated with a suitable catalyst.

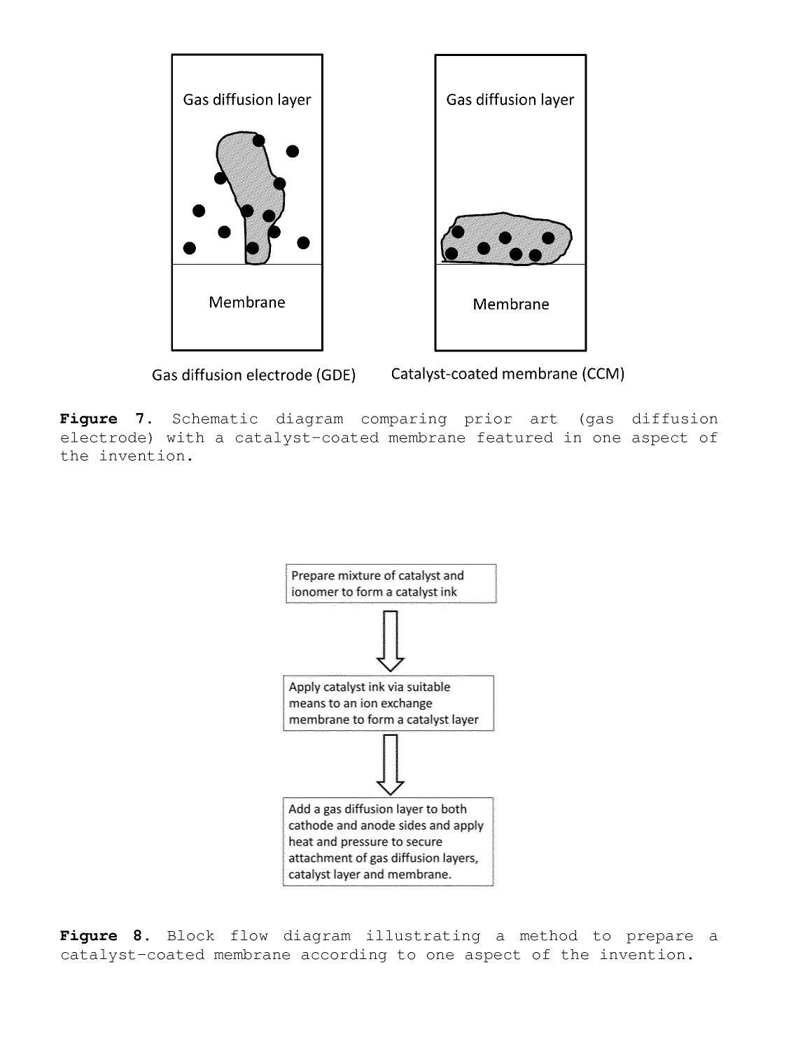

In another aspect, the cathode electrode consists of a catalyst coated membrane, as seen in FIG. 7. This ensures the catalyst layer is in immediate contact with the membrane, and it avoids dispersion of the catalyst ink into the gas diffusion layer. This is preferred because the catalyst itself may decompose hydrogen peroxide after it has been extracted. Limiting the catalyst layer at the region neighboring the membrane minimizes this effect. Suitable catalyst materials include those known for oxygen reduction to hydrogen peroxide, such as silver, carbon, gold, Pt—Au, Pd—Au, Pt—Hg, Pd—Hg, Ag—Hg, Cu—Hg, Co-containing materials, graphene or other carbon-based structures, graphene doped with transition or post-transition metals, porphyrins containing transition metals or post-transition metals or any combinations thereof.

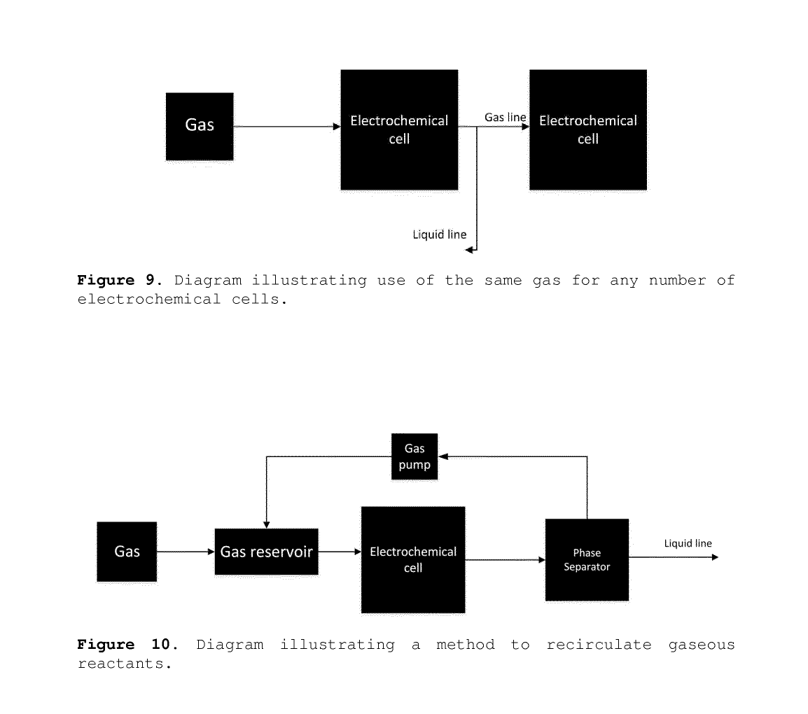

In another aspect, the oxygen-containing gas is delivered to the cathode and exits the housing through an outlet. At this point the gas may be directed back into the cathode of the same cell, or to the cathode of another cell, as described in FIG. 9. This is particularly beneficial when using a gas with a higher concentration of oxygen than air (for example gas obtained from an oxygen concentrator). This enables utilization of any oxygen which was not used in the initial electrochemical cell. If there is only one cell, gas can be accumulated in a gas tank, fed into the cell and once it exits the cell recirculated into the gas tank from which it can be reintroduced into the cell, as shown in FIG. 10. This same method can also be utilized when there are several electrochemical cells, in a manner that gas is fed into the first cell, exits the first cell, and it enters the second cell, from where it is directed into the third cell or recirculated into the gas tank. A phase separator to separate water and gas may be utilized between the two cells. This method can be utilized for an indefinite number of cells, the only limiting factor being the pressure of the gas.

The oxygen gas produced at the anode of the electrochemical cells can also be merged with the cathode gas feed at any point of the gas lines. This may enable even higher oxygen concentrations at the cathode gas feeds.

In an example cell, the cathode is made of an ink consisting of a suitable catalyst, ionomer solution, a suitable solvent (typically an alcohol) and water. The solid content of the ink has a catalyst ratio of between 1:5 to 4:5, an ionomer ratio between 1:5 to 4:5 and may contain PTFE in ratios between 0 to 4:5, preferably between 1:20 to 4:5. The ink may also contain a suitable surfactant, such as Triton, a quaternary ammonium compound or other suitable polymers in ratios between 0 to 4:5. The ink is applied onto a gas diffusion layer (with the resulting electrode known as gas diffusion electrode). Further, the coating can take place on one side or on both sides of the gas diffusion layer. Alternatively, the cathode can also consist of a catalyst coated membrane, where the ink is sprayed directly on an ion exchange membrane and a gas diffusion layer is added afterwards.

The anode in this example is a water oxidation anode, and is prepared in the catalyst coated membrane configuration well-known to those versed in the art (see for example Handbook of Fuel Cells: Fundamentals, Technology and Applications, Wiley VCH, 2014). Other anode reactions than water oxidation, including suitable materials, could be used without affecting the present example.

Anode and cathode are pressed on either side of an ion exchange membrane and incorporated into the electrochemical cell housing. The compartments of the housing are filled with water, and an oxygen-containing gas is injected into the cathode side of the system. In this example the distance between the gas disperser and the cathode gas diffusion layer is <1 mm. The experiment takes place at room temperature, and the pressure of the gas can vary between 0 and 50 bar, preferably between 0 and 5 bar. In a typical experiment a potential difference between 0.5 and 2.5 V, preferably between 1.5 and 2.2 V, is applied between anode and cathode, and current is observed indicating electrochemical reactions are taking place. Preferably the current is above 30 mA/cm², and even more preferably above 50 mA/cm². The experiment is let run for a certain amount of time, while water is flowing through both anode and cathode compartments, and the hydrogen peroxide concentration produced at the cathode is determined via a suitable method, which could be permanganate titration or indicator strips. This hydrogen peroxide can be accumulated in a reservoir for its later use, or utilized directly.

1.-16. (canceled)

17. An apparatus for producing a chemical compound by electrochemical action, comprising:
an anode electrode;
an anode catalyst layer;
an ion exchange membrane;
wherein the anode catalyst layer is in physical contact with the membrane on a first side of the membrane and the anode electrode is in physical contact with the anode catalyst layer on the side of the anode catalyst layer not in contact with the membrane;
a cathode electrode;
a cathode catalyst layer;
wherein the cathode catalyst layer is in physical contact with the membrane on a second side of the membrane and the cathode electrode is in physical contact with the cathode catalyst layer on the side of the cathode catalyst layer not in contact with the membrane;
a gas diffusion layer (GDL) in contact with the side of the anode electrode not in contact with the anode catalyst layer;
a gas diffusion layer (GDL) in contact with the side of the cathode electrode not in contact with the cathode catalyst layer;
wherein one or both of the anode electrode and the cathode electrode are at least partially immersed in an aqueous solution liquid;
one or more gas dispersers, the one or more gas dispersers being spaced from the GDLs within a distance of 1 cm, the aqueous solution liquid being between the GDLs and the one or more gas dispersers; and,
a gas reactant feed to one or both of the anode and cathode.

18. The apparatus with the gas disperser of claim 17 wherein the gas is directed into the gas diffusion layer (GDL) and through to a catalyst layer .

19. The apparatus of claim 17 wherein the chemical compound is electrochemically synthesized hydrogen peroxide produced by oxygen reduction at the cathode side of the cell.

20. The apparatus of claim 19 wherein an oxygen source is air, oxygen extracted from air or an oxygen supply.

21. The apparatus of claim 17 wherein the aqueous solution liquid is made to flow.

22. The apparatus of claim 17 wherein the cathode is a gas diffusion electrode.

23. The apparatus of claim 22 wherein the cathode catalyst is chosen from the group including silver, carbon, gold, Pt—Au, Pd—Au, Pt—Hg, Pd—Hg, Ag—Hg, Cu—Hg, Co-containing materials, graphene or other carbon-based structures, graphene doped with transition or post-transition metals, porphyrins containing transition metals or post-transition metals or any combinations thereof.

24. The apparatus of claim 17 wherein the anode catalyst is chosen from the group including iridium, ruthenium, platinum, gold or mixtures thereof and is applied as a catalyst-coated membrane.

25. The apparatus of claim 17 wherein the membrane is coated with a catalyst material for cathode reaction forming a catalyst coated membrane.

26. The apparatus of claim 25 wherein the cathode catalyst is selected from the group including silver, carbon, gold, Pt—Au, Pd—Au, Pt—Hg, Pd—Hg, Ag—Hg, Cu—Hg,Co-containing materials, graphene or other carbon-based structures, graphene doped with transition or post-transition metals, porphyrins containing transition metals or post-transition metals or any combinations thereof.

27. The apparatus of claim 17 wherein the gas supplied to the electrochemical cell contains oxygen, and oxygen is fed into the electrochemical cell and recirculated for its utilization in the same or other cells which may be connected in parallel or in series.

28. The apparatus of claim 19 wherein the electrochemically synthesized hydrogen peroxide is introduced locally into a water line or a reservoir.

29. The apparatus according to claim 28, wherein the synthesized hydrogen peroxide is vaporized in order to provide air and surface disinfection.

30. The apparatus according to claim 28, wherein synthesized hydrogen peroxide is combined with ultra-violet light or ozone in order to facilitate the formation of hydroxyl radicals.

31. Method for producing a chemical compound by electrochemical means using one or more electrochemical cells comprising:
providing an anode electrode;
providing an anode catalyst layer;
providing an ion exchange membrane;
wherein the anode catalyst layer is in physical contact with the membrane on a first side of the membrane and the anode electrode is in physical contact with the anode catalyst layer on the side of the anode catalyst layer not in contact with the membrane;
providing a cathode electrode;
providing a cathode catalyst layer;
wherein the cathode catalyst layer is in physical contact with the membrane on a second side of the membrane and the cathode electrode is in physical contact with the cathode catalyst layer on the side of the cathode catalyst layer not in contact with the membrane;
providing a gas diffusion layer (GDL) in contact with the side of the anode electrode not in contact with the anode catalyst layer;
providing a gas diffusion layer (GDL) in contact with the side of the cathode electrode not in contact with the cathode catalyst layer;
at least partially immersing one or both of the anode electrode and the cathode electrode in an aqueous solution liquid;
providing one or more gas dispersers, the one or more gas dispersers being spaced from the GDLs within a distance of 1cm;
providing the aqueous solution liquid between the GDLs and the one or more gas dispersers; and,
feeding a reactant gas through the liquid directly to the cathode or anode or to both sides of the cell.

32. The method of claim 31, further comprising directing the gas is directed into the gas diffusion layer (GDL) and through to a catalyst layer.

33. The method of claim 31 wherein the chemical compound is electrochemically synthesized hydrogen peroxide produced by oxygen reduction at the cathode side of the cell.

34. The method of claim 33, further comprising introducing the electrochemically synthesized hydrogen peroxide locally into a water line or a reservoir.

35. The method of claim 34, further comprising vaporizing the synthesized hydrogen peroxide in order to provide air and surface disinfection.

36. The apparatus according to claim 33, further comprising the step of combining the synthesized hydrogen peroxide with ultra-violet light or ozone in order to facilitate the formation of hydroxyl radicals.
